SEQUENCE LISTING (3 sequences)

SEQ ID NO: 1 - DNA, 20 bp, Artificial Sequence
  PRIMER
tatagaggctcaggaaaaag

SEQ ID NO: 2 - DNA, 20 bp, Artificial Sequence
  primer
atgcaatgcaagcctgagct

SEQ ID NO: 3 - DNA, 20 bp, Artificial Sequence
  reverse primer
tatagaggctcaggaaaaag

(19) United States
(12) Patent Application Publication
(10) Pub. No.: US 2012/0208873 A1
(43) Pub. Date: Aug. 16, 2012

(54) TEA POLYPHENOLS PRODUCTS FOR CEASING SMOKING AND TREATING AND/OR PREVENTING NICOTINE OR NICOTINE-DERIVED COMPOUNDS OR ESTROGEN INDUCED BREAST CANCER

(75) Inventors: Shih-Hsin Tu, Taipei City (TW); Chung-Yu Ku, Taipei City (TW); Ching-Shyang Chen, Taipei City (TW); Ching-Shui Huang, Taipei City (TW); Chia-Hwa Lee, Taipei City (TW); Li-Ching Chen, Taipei City (TW); Min-Hsiung Pan, Kaohsiung City (TW); Hui-Wen Chang, Taipei City (TW); Chien-Hsi Chang, Taipei City (TW); Yu-Jia Chang, Taipei City (TW); Po-Li Wei, Taipei City (TW); Chih-Hsiung Wu, Taipei County (TW); Yuan-Soon Ho, Taipei City (TW)

(73) Assignee: TAIPEI MEDICAL UNIVERSITY, Taipei City (TW)

(21) Appl. No.: 13/118,919

(22) Filed: May 31, 2011

Related U.S. Application Data
(63) Continuation-in-part of application No. 13/025,401, filed on Feb. 11, 2011.

(51) Int. Cl. A61K 31/353; A61P 35/00

(52) U.S. Cl. 514/456

(57) ABSTRACT

This invention relates to methods of aiding smoking cessation and/or treating and/or preventing smoking addition or methods of treating and/or preventing nicotine or nicotine-derived compounds or estrogen induced breast cancer, which comprises administering to a subject an effective amount of a tea polyphenol. The invention uses catechins (such as epigallocatechin gallate (“EGCG”), epigallocatechin (“EGC”), epicatechin gallate (“ECG”), epicatechin (“EC”), gallocatechin gallate (“GCG”), gallocatechin (“GC”), catechin gallate (“CG”) and catechin (“C”)) in aiding smoking cessation and/or treating and/or preventing smoking addition or treating and/or preventing nicotine or nicotine-derived compounds or estrogen induced breast cancer.

### CROSS-REFERENCE TO RELATED APPLICATIONS

This application claims priority to, and is a Continuation in part of, U.S. patent application Ser. No. 13/025,401, filed on Feb. 11, 2011, which is hereby incorporated by reference in its entirety.

### FIELD OF THE INVENTION

This invention relates to methods of aiding smoking cessation and/or treating and/or preventing smoking addition or methods of treating and/or preventing nicotine or nicotine-derived compounds or estrogen induced breast cancer, which comprises administering to a subject an effective amount of a tea polyphenol. In particular, the invention uses catechins in aiding smoking cessation or treating and/or preventing nicotine or nicotine-derived compounds or estrogen induced breast cancer.

### BACKGROUND OF THE INVENTION

Breast cancer is the second leading cause of cancer-related deaths among women in the United States. Large cohort studies performed in the United States and Japan have indicated that the risk of developing breast cancer is associated with active and passive smoking. Cigarette smoke is a complex mixture of over 4000 chemical constituents and contains nicotine (Nic) as a major carcinogenic component. Studies performed using ¹⁴C-Nic showed that 80-90% of inhaled Nic is absorbed systemically during smoking (Armitage, A. K., Dollery, C. T., George, C. F., Houseman, T. H. et al., Br. Med. J. 1975, 4, 313-316). Nic concentrations in plasma average about 15 ng/mL immediately after smoking and are extremely high in the saliva and gastric juices (>1300 and >800 ng/mL, respectively). Repeated exposure of noncancerous human breast epithelial (MCF-10A) cells to the tobacco-specific carcinogen 4-(methylnitrosamino)-1-(3-pyridyl)-1-butanone (NNK) results in xenografted tumor formation in nude mouse models and anchorage-independent colony formation in soft agar. In vivo studies have demonstrated that Nic promotes the growth of solid tumors, suggesting that Nic might contribute to the progression of cell proliferation, invasion, and angiogenesis in tumors (Heeschen, C., Jang, J. J., Weis, M., Pathak, A. et al., Nat. Med. 2001, 7, 833-839). These results imply that Nic alteration of normal breast epithelial cells may also contribute to breast tumorigenesis.

Estrogen stimulates breast and endometrial tissue proliferation, enhances bone density, and lowers plasma cholesterol. Selective estrogen receptor modifiers (SERMs). SERMs are small ligands of the estrogen receptor that are capable of inducing a wide variety of conformational changes in the receptor, thereby eliciting a variety of distinct biological profiles. Many SERMs are bifunctional in that they antagonize some of these functions while stimulating others. For example, tamoxifen, which is a partial agonist/antagonist at the estrogen receptor, inhibits estrogen-induced breast cancer cell proliferation but stimulates endometrial tissue growth and prevents bone loss.

Human neuronal tissues have been reported to have a high level of nicotinic acetylcholine receptor (nAChR) subunit expression upon chronic Nic exposure. The natural ligand of nAChRs is acetylcholine; however, tobacco components, such as Nic and NNK, are known to be high-affinity agonists of nAChRs. The previous reports indicate that cigarette smoking is the chief risk factor in lung, colon, bladder, and breast cancers. Animal experiments have demonstrated that overexpression of cyclin D3 is detected concomitantly in a9-nAChR-overexpressing MCF-10A-Nic (DOX)-xenografted tumor tissues in BALB/c-nu/nu mice (Chen, C. S., Lee, C. H., Hsieh, C. D., Ho, C. T. et al., Breast Cancer Res. Treat. 2010). It has further been demonstrated that agents that were isolated from natural edible plants could inhibit human breast cancer cell proliferation by specifically targeting human nAChRs (Shih, Y L., Liu, H. C., Chen, C. S., Hsu, C. H. et al., J. Agric. Food Chem. 2010, 58, 235-241; Lin, Y. S., Tsai, Y. J., Tsay, J. S., Lin, J. K., J. Agric. Food Chem. 2003, 51, 1864-1873). These results imply that nAChRs detected in human breast cancer cells could be used as a therapeutic molecular target for clinical applications.

It has been known that tea polyphenols have various effects such as an antioxidating effect (U.S. Pat. No. 4673530 and Japanese Patent Laid-open No. H1-268683), antimicrobial and bacteriostatic effects (Japanese Patent Laid-open No. H2-276562 and EPO443090 B1), an inhibitory activity for cholesterol increase (Japanese Patent Laid-open No. S60-156614), an inhibitory activity for blood pressure increase (U.S. Pat. No. 4840966 B1) and an inhibitory activity for blood glucose increase (EP 0573682 A1). Moreover, the green tea extract (−)-epigallocatechin-3-gallate (EGCG) had been studied for its antioxidant and free radical scavenging activity (Lin, Y. S., Tsai, Y. J., Tsay, J. S., Lin, J. K., J. Agric. Food Chem. 2003, 51, 1864-1873; Lin, J. K, Chen, P. C., Ho, C. T., Lin-Shiau, S. Y., J. Agric. Food Chem. 2000, 48, 2736-2743). EGCG is also effective in inducing breast cancer apoptosis and cell-cycle arrest (Pan, M. H., Lin, C. C., Lin, J. K, Chen, W. J., J. Agric. Food Chem. 2007, 55, 5030-5037; Stuart, E. C., Rosengren, R. J., Life Sci. 2008, 82, 943-948). Another study has also suggested that many of the cancer chemopreventive properties of green tea are mediated by EGCG, which induces apoptosis and promotes cell growth arrest by altering the expression of cell-cycle regulatory proteins, activating killer caspases, and suppressing nuclear factor xB activation (Butt, M. S., Sultan, M. T., Crit. Rev. Food Sci. Nutr. 2009, 49, 463-473).

However, none of prior art discloses or suggests the relationship of tea polyphenols to nicotine or nicotine-derived compounds or estrogen induced breast cancer.

### SUMMARY OF THE INVENTION

One object of the invention is to provide a method of treating and/or preventing nicotine or nicotine-derived compounds or estrogen induced breast cancer, which comprises administering to a subject an effective amount of a tea polyphenol or a pharmaceutically acceptable salt thereof, thereby treating and/or preventing nicotine or nicotine-derived compounds or estrogen induced breast cancer. The invention also provides a new use of tea polyphenol or a pharmaceutically acceptable salt thereof in the manufacture of a medicament for treating and/or preventing nicotine or nicotine-derived compounds or estrogen induced breast cancer.

Another object of the invention is to provide a method of aiding smoking cessation and/or treating and/or preventing smoking addition, which comprises administering to a subject an effective amount of a tea polyphenol or a pharmaceutically acceptable salt thereof, thereby aiding smoking cessation and/or treating and/or preventing smoking addition. The invention also provides a new use of tea polyphenol or a pharmaceutically acceptable salt thereof in the manufacture of a medicament for aiding smoking cessation and/or treating and/or preventing smoking addition.

### BRIEF DESCRIPTION OF THE DRAWING

FIG. 1 shows Nic- and E2-induced nicotinic receptor upregulation in human breast cancer cells. (A) MCF-7 cells were treated with Nic (0.1-10 μM), NNK (0.1-10 μM), or E2 (1-200 nM) for 24 h. The α9-nAChR protein levels were determined by immunoblotting analysis. (B) Human breast cancer cells with (MCF-7 and BT483) or without (MDA-MB-231 and MDA-MB-453) ER expression were treated with E2 (10 nM) for 24 h. The α9-nAChR protein levels were determined by immunoblotting analysis. (C) MCF-7 cells were treated with Nic (1 μM) for various times. The α9-nAChR mRNA (upper panel) and protein (lower panel) levels were determined by RT-PCR and immunoblotting, respectively. The α9-nAChR mRNA expression level was measured and normalized to β-glucuronidase expression using the Roche LightCycler Software Version 4, as described in a previous report (Chen, C. S., Lee, C. H., Hsieh, C. D., Ho, C. T. et al., Breast Cancer Res. Treat. 2010). The mRNA or protein changes were analyzed by densitometry (Bio-1D chemiluminescent imaging system) and the results of differences were calculated by statistical methods. Results are representative of at least three independent experiments.

FIG. 2 shows that Nic- and E2-induced α9-nAChR expression is inhibited by EGCG in human breast tumor cell lines. (A) MCF-7 cells were treated with Nic (10 μM) in the presence or absence of natural polyphenolic compounds (1 μM, each compound) for 24 h. Expression of a9-nAChR mRNA was then detected by RT-PCR analysis. (B) MCF-7 cells were treated with Nic (10-μM) or E2 (10 nM) in the presence or absence of EGCG (1-10-μM) for 24 h. The α9-nAChR protein was then detected by immunoblotting. Results are representative of at least three independent experiments. (C) MCF-7 cells were treated with Nic (10 μM) in the presence or absence of EGCG (1-10 μM) for 24 h. The α9-nAChR mRNA expression levels were evaluated by real-time PCR analysis. Data are the mean±SE of three independent experiments. Significance was set at p<0.05.

FIG. 3 shows the effect of EGCG on Nic- and E2-induced PI3K activity in MCF-7 cells. (A, B) MCF-7 cells were pretreated with a PI3K/AKT inhibitor (LY294002, 25 μM) or EGCG (10 μM) for 24 h and subsequently treated with (A) E2 (10 nM) or (B) Nic (10 μM) for an additional 24 h. The α9-nAChR, phosphorylated AKT (p-AKT), and total AKT proteins were then detected by immunoblotting.

FIG. 4 shows AP-1 mediates α9-nAChR transcriptional regulation by Nic and E2 in MCF-7 cells. (A) Schematic representation of the a9-nAChR promoter region (−1000/−1) illustrates the putative AP1, and p53 transcription factor binding sites. (B) MCF-7 cells were transiently transfected with full-length (−1000 to −1) pGL3 (α9-nAChR) and pRL-TK plasmid for 24 h. These cells were pretreated with EGCG (10 μM) for 24 h and then treated with Nic (10 μM) or E2 (10 nM) for an additional 24 h. Cell lysates were harvested, and relative firefly luciferase activities were measured and normalized to renilla luciferase from the same cell lysates. Luciferase activity obtained from the control cells treated only with vehicle (0.1% DMSO for E2 and ddH2O for Nic) treatment was defined as a one-fold change. Data were analyzed using paired t-tests; all p-values are two sided. The comparisons were carried out as follows: the Nic- (or E2-) treated groups were compared between vehicle, EGCG+E2, or EGCG+Nic treatment groups (*p<0.05). (C) MCF-7 cells were transiently transfected with different length pGL3 (α9-nAChR) promoters and pRL-TK plasmids for 24 h. The transfected cells were pretreated with EGCG (10 μM) for 24 h and then treated with Nic (10 μM) for an additional 24 h. Data were analyzed using paired t-tests; all p-values are two sided. The comparisons were carried out as follows: the Nic-treated groups were compared with the vehicle and EGCG+Nic combined treatment groups (*p<0.05). (D) MCF-7 cells were treated with Nic (10 μM) for 24 h or pretreated with EGCG (10 μM), as described above. After treatment, the cell lysates were harvested and DNA bound by AP-1 or p53 was precipitated using specific antibodies for ChIP. To avoid false-negative results, immune-precipitation experiments were performed using antibody specific for p53. The p53 protein was detected by a p53-specific antibody (right panel). Data shown are representative of three independent experiments with similar results. Genomic DNA isolated from MCF-7 was used as a positive input control to ascertain the PCR conditions.

FIG. 5 shows the effects of EGCG on the inhibition of Nic-induced breast cancer cell proliferation through blocking Nic binding to α9-nAChR. (A) MCF-7 and MCF-10A cells were treated with Nic (10 μM, 24 h) or vehicle alone, with or without EGCG pretreatment (10 μM, 24 h). Cell growth was measured by the MTT assay at 24 h after drug treatment. (B) MCF-7, α9-nAChR (Si), and (Sc) cells were exposed to EGCG (10 μM) for 24 h then treated with Nic (10 μM) for an additional 24 h. Cell proliferation was assayed with MTT at the indicated times. (C) MCF-7 cells were pretreated with EGCG (10 μM) for 24 h. After treatment, [³H]-Nic was added to the EGCG-exposed cells for an additional 24 h (EGCG pretreated group). In addition, the EGCG-treated cells were washed with PBS three times, and [³H]-Nic was then added for an additional 24 h (EGCG pretreated then wash group). The cells that were treated with both EGCG and [³H]-Nic for 24 h are designated as the EGCG cotreated group. The specific binding activity is presented as: total binding—nonspecific binding. (C) The α9-nAChR (Si), (Sc), and wild-type MCF-7 (C) cells were pretreated with EGCG (10 μM) for 24 h. After EGCG pretreatment, the cells were then treated with [³H]-Nic (10 μM) for an additional 24 h and the [³H]-Nic binding activity was then evaluated. Data are the mean±SE of three independent experiments. Significance was set at *p<0.05.

FIG. 6 shows effects of EGCG on the inhibition of Nic-induced breast cancer (MCF-7) cell colony formation in soft agar. (A) MCF-7 cells were treated with Nic (10 μM) or vehicle alone with or without EGCG (10 μM). The gross morphology of MCF-7 cell colonies on culture plates is shown. These colonies exhibit subtle changes in their morphology, including a slight disaggregation compared with the untreated cells. Bar=200 μm. (B) The number of colonies scored from the soft agar plates: the wild-type MCF-7. (C), α9-nAChR (Si), and (Sc) cells were seeded in soft agar and treated with Nic in the presence or absence of EGCG. The colonies in a 1×3 cm area were counted on each plate. Data are the mean±SE of three independent experiments. Significance was set at p<0.05. *The combined EGCG+Nic-treated groups were significantly different from the Nic-treated groups. #The combined EGCG+Nic-treated groups in the siRNA cells were significantly different from the EGCG+Nic-treated groups in the wild type (C) and (Sc) MCF-7 cells.

FIG. 7 shows a schematic diagram of PI3K-mediated AP-1 transcriptional activation of the α9-nAChR gene promoter in MCF-7 cells and Nic administration to human MCF-7 cells induces PI3K activation. The subsequently activated kinases (such as pAKT) induce the activation of AP-1, which then translocates to the nucleus in a concerted fashion, resulting in binding to the AP-1 sites of the α9-nAChR promoter. Expression of the α9-nAChR protein is thus induced, leading to amplification of the Nic-induced carcinogenic signals, which then lead to increased MCF-7 cell proliferation.

### DETAILED DESCRIPTION OF THE INVENTION

The invention surprisingly found that the nicotinic receptor α9-nAChR is significantly induced by Nic and estradiol (E2) in human breast cancer and that E2- and Nic- or Nic-derived compounds induced α9-nAChR protein expression is blocked by tea polyphenols. The invention also unexpectedly found that tea polyphenols can be used as an agent for blocking smoking (nicotine, Nic) or hormone (estradiol, E2)-induced breast cancer cell proliferation by downregulating α9-nAChR expression. In view of the above-mentioned mechanism, since tea polyphenols can block the expression of nicotinic receptor, they can be used in aiding smoking cessation. Therefore, the invention suggests a smoking cessation ability of tea polyphenols, or a chemopreventive ability of tea polyphenols through the inhibition of E2- or Nic or Nic-derived compounds induced α9-nAChR protein expression, which confers smoking-mediated breast tumorigenesis.

The terms “a” and “an” refer to one or to more than one (i.e., to at least one) of the grammatical object of the article.

The term “treat,” “treatment” or “treating” means reducing the frequency, extent, severity and/or duration with which symptoms of breast cancer are experienced by a patient.

The term “prevent,” “prevention” or “preventing” means inhibition or the averting of symptoms associated with breast cancer.

The term “alleviate,” “alleviation” or “alleviating” means reducing the severity of one or more symptoms of a disease, disorder or condition.

The term “tea polyphenol” (hereinafter referred to as “TP”) refers to the major water-soluble substances in tea, accounting for 10% of the dry weight of tea to 25%. TP mainly includes four substances: catechins, flavonoids, anthocyanins, phenolic acids and phenolic acids reduced. Of which the most abundant catechins, about 60% of the total to 80%.

The term “catechin” is a collective term including epigallocatechin gallate (hereinafter referred to as EGCG), epigallocatechin (hereinafter referred to as EGC), epicatechin gallate (hereinafter referred to as ECG), epicatechin (hereinafter referred to as EC), gallocatechin gallate (hereinafter referred to as GCG), gallocatechin (hereinafter referred to as GC), catechin gallate (hereinafter referred to as CG) and catechin (hereinafter referred to as C). Each of the catechins described here may be either (+)-form or (−)-form, and (−)-EGCG, (−)-EGC, (−)-ECG, (−)-EC, (−)-GCG, (−)-GC, (−)-CG and (+)-C are preferable.

The term “pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

The term “effective amount” means an amount of tea polyphenol effective to treat and/or prevent nicotine or nicotine-derived compounds or estrogen induced breast cancer. For example, the effective amount of the tea polyphenol may reduce the number of cancer cells; reduce the tumor size; inhibit (i.e., slow to some extent and preferably stop) cancer cell infiltration into peripheral organs; inhibit (i.e., slow at least to some extent and preferably stop) tumor metastasis; inhibit, to some extent, tumor growth; promote apoptosis; and/or relieve to some extent one or more of the symptoms associated with the disorder. In addition, in the respect of smoking cessation, the “effective amount” means the amount sufficient to aid smoking cessation.

Methods of Treating and/or Preventing Nicotine or Nicotine-Derived Compounds or Estrogen Induced Breast Cancer

In one aspect, the invention provides a method of treating and/or preventing nicotine or nicotine-derived compounds or estrogen induced breast cancer, which comprises administering to a subject an effective amount of a tea polyphenol or a pharmaceutically acceptable salt thereof, thereby treating and/or preventing nicotine or nicotine-derived compounds or estrogen induced breast cancer.

It is known in the art that nicotine is not a complete carcinogen and nitrosation of nicotine gives NNN (“N′-nitrosonomicotine”) by cleavage of the N—CH₃ bond with loss of formaldehyde or yields NNK (“4-(methylnitros-amino}-t-(3-pyridyl)-1-butanone” (the origin of the term NNK is “nicotine-derived nitrosaminoketone”) or NNA (“4-fmethylnitrosamino)-4-{3-pyridyl)-butanal”) by cleavage of either the 2′-N or 5′-N bond, respectively (Cancer Research 45, 935-944, March 1985, which incorporated herein by reference in its entirety). The nitotine derived compounds are carcinogens.

According to the invention, various tea polyphenols (TP) can be used as the active ingredient in the methods of the invention. In one embodiment, the TP are catechins. Preferably, the catechins include, but are not limited to, epigallocatechin gallate (EGCG), epigallocatechin (EGC), epicatechin gallate (ECG), epicatechin (EC), gallocatechin gallate (GCG), gallocatechin (GC), catechin gallate (CG) and catechin (C). The above-mentioned compounds may be in either (+)-form or (−)-form. Preferably, the catechin is EGCG. More preferably, the catechin is (−)-EGCG.

According to the invention, suitable pharmaceutically acceptable salts of TP include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

According to the invention, TP or a pharmaceutically acceptable salt thereof is useful to treat and or prevent conditions that are associated with nicotine or nicotine-derived compounds or estrogen induced breast cancer. In particular, nicotine or nicotine-derived compounds or estrogen induced breast cancer is caused by E2- or Nic-induced expression (especially overexpression) of the α9-nAChR protein. According to the invention, TP or a pharmaceutically acceptable salt thereof confers smoking-mediated breast tumorigenesis and chemopreventive ability. TP or salts thereof may optionally be used in combination therapies to treat any of these diseases or conditions. The combinations include TP or its salt combined with surgery, radiation therapy, chemotherapy, cytotoxic agents, cytostatic agents or hormone therapies, including any of the therapies or treatments disclosed herein or in any of the references cited herein. Preferably, the TP can be used in combination with chemotherapy.

An effective dose of TP or its salts for use in therapeutic applications, e.g., nicotine or nicotine-derived compounds or estrogen induced breast cancer, will depend to a certain extent at least on factors such as the status of the condition being treated, whether TP or its salt is being used prophylactically (lower doses) or the severity of the malignancy, the method of delivery, and the pharmaceutical formulation. These factors will be determined by the clinician using conventional dose escalation studies. Typically the dose administered to the subject will be from about 500-2,500_mg/kg body weight. Preferably, the TP dose is about 500-2,000, 700-2,000, 1000-2000, 1000-1800 or 1200-1600. More preferably, the TP does is 1500_mg/kg body weight.

Methods of Aiding Smoking Cessation and Treating and/or Preventing Smoking Addiction

In another aspect, the invention provides a method of aiding smoking cessation and/or treating and/or preventing smoking addition, which comprises administering to a subject an effective amount of a tea polyphenol or a pharmaceutically acceptable salt thereof, thereby aiding smoking cessation and/or treating and/or preventing smoking addition.

According to the invention, the TP or a pharmaceutically acceptable salt thereof used in the method of aiding smoking cessation and/or treating and/or preventing smoking addition is that as mentioned herein.

The World Health Organization (WHO) classifies smoking and nicotine addiction as one of the most prevalent addictive human behavior worldwide. In spite of increased awareness and public action, it remains the leading cause of preventable disease and death. As one skilled in the art will readily appreciate, the act of smoking involves both physiological responses, e.g., nicotine-induced biochemical signals in the brain, and sensory and psychological signals, e.g., smell, taste, the ritualization of smoking. Over time, the reinforcing activity of nicotine becomes closely linked to the sensory and psychological responses associated with smoking. As a result, the design of methods of assisting smokers to cease smoking preferably allows nicotine reinforcement to be dissociated from the sensory and psychological aspects of smoking.

Accordingly, the invention is directed to a method of aiding smoking addiction. This may be accomplished by administering a tea polyphenol or a pharmaceutically acceptable salt thereof to a subject in need of ceasing smoking.

Accordingly, the invention is also directed to a method of treating smoking addiction. This may be accomplished by administering a tea polyphenol or a pharmaceutically acceptable salt thereof to a subject in need of terminating a smoking addiction. While not wishing to bound by any particular theory, it is believed that by smoking addiction may be successfully treated by blocking some of the pharmacological effects of nicotine, such as, but not limited to reinforcement, antinociception, hypothermia, drug discrimination and motor impairment, while also dissociating some of the reinforcing affects of smoking. Within the context of the present invention, a subject in need of terminating a smoking addiction is a person who smokes on a regular basis and is either unable or unwilling to terminate smoking on a regular basis. The method of treating a smoking addiction may be practiced, by administering TP of the present invention or a pharmaceutically acceptable salt thereof as described, preferably concurrent with or in advance of the act of smoking. In this fashion, the subject addicted to smoking will also be subject to the effects of TP or a pharmaceutically acceptable salt thereof while smoking, which can act to dissociate the reinforcing effects of smoking, from the act of smoking itself. The amount of TP or a pharmaceutically acceptable salt thereof administered to be effective to dissociate the reinforcing effects of smoking from the act of smoking may vary depending on the subject and the nature of the subject addiction to smoking, however, determination of effective dosages and treatment schedules is within the level of skill of those of ordinary skill in the art, without undue experimentation.

Accordingly, the present invention is also directed to a method of preventing an addiction to smoking, by administering TP or a pharmaceutically acceptable salt thereof. A subject in need of preventing an addiction to smoking may be a non-smoker or an occasional smoker, who is concerned about developing an addiction to smoking. The method of preventing a smoking addiction may be practiced, by administering TP or a pharmaceutically acceptable salt thereof as described, preferably in advance of the act of smoking. In this fashion, the patient will not develop a strong association of the act of smoking with the reinforcing effects of smoking. The amount of TP or a pharmaceutically acceptable salt thereof administered to be effective to prevent the association of the reinforcing effects of smoking from the act of smoking may vary depending on the patient and the nature of the patient. However, determination of effective dosages and treatment schedules is within the level of skill of those of ordinary skill in the art, without undue experimentation.

### Formulations and Administration of Tea Polyphenol or its Salt

Any suitable route of administration may be employed for providing the patient with an effective dosage of TP or a pharmaceutically acceptable salt thereof. For example, oral, rectal, parenteral, transdermal, transmucosal, subcutaneous, intramuscular, intrathecal, intrapulmonary, nasal or vaginal and the like may be employed as appropriate. Dosage forms include tablets, coated tablets, caplets, capsules, troches, dispersions, suspensions, solutions, patches, lozenges, gums, suppository, ointment, gel, pessary, membrane, powder and the like, including sustained release formulations well known in the art.

Pharmaceutical formulations that comprise TP or its salt will typically comprise one or more carriers or excipients and optionally other therapeutic ingredients. The carrier(s) will generally be “acceptable” in the sense of being compatible with the other ingredients of the formulation and physiologically innocuous to the recipient thereof. Such carriers or excipients are known, e.g., fillers, lubricants, binders and various liquid excipients for liquid formulations. Suitable carriers include those disclosed in the references cited herein.

Suitable formulations include aqueous or oily solutions of TP or its salt. Formulations suitable for parenteral delivery of the active ingredient include aqueous and non-aqueous compositions where the active ingredient is dissolved or suspended in solution. Formulations suitable for parenteral administration include aqueous and non-aqueous sterile injection solutions which may contain anti-oxidants, buffers, bacteriostats or solutes that render the formulation isotonic with the blood of the intended recipient. Other parenteral formulations may comprise aqueous and non-aqueous sterile suspensions which may include suspending agents and thickening agents.

Formulations of the invention suitable for oral administration are prepared as discrete units such as capsules, cachets, gums or tablets each containing a predetermined amount of TP or its salt; as a powder or granules; as solution or a suspension in an aqueous liquid or a non-aqueous liquid; or as an oil-in-water liquid emulsion or a water-in-oil liquid emulsion.

Formulations for rectal administration may be presented as a suppository with a suitable base.

Formulations suitable for intrapulmonary or nasal administration will have a particle size, for example, in the range of 0.01 to 200 microns (including particle sizes in a range between 0.01 and 500 microns in increments of 0.1 microns such as 0.1, 0.2, 0.3, 0.4, 0.5, 1, 2, 5, 30 microns, 35 microns, etc.), which is administered by inhalation through the nasal passage or by inhalation through the mouth so as to reach the various bronchi or alveolar sacs. Formulations suitable for aerosol or dry powder administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for transdermal administration may be presented as transdermal patches. The transdermal patch provides a base line or steady state nicotine level to the patient. The total amount of TP or a pharmaceutically acceptable salt thereof released by the patch during the period of use will vary depending on the user's body size, history of exposure to nicotine, and response to treatment. The size of the patch will vary according to the amount of nicotine to be delivered.

Formulations comprising TP or its salt are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as described herein, or an appropriate fraction thereof, of the active ingredient.

It should be understood that in addition to TP or its salts particularly mentioned above the formulations of this invention may include other agents conventional in the art having regard to the type of formulation in question, for example those suitable for oral administration may include flavoring or coloring agents.

TP or its salt may be used to provide controlled release pharmaceutical formulations containing TP or its salt in which the release of TP or its salt is controlled and regulated to allow less frequent dosing or to improve the pharmacokinetic or toxicity profile of TP or its salt.

The formulations include those suitable for any of the foregoing administration routes. The formulations may conveniently be presented in unit dosage form and may be prepared by any of the methods well known in the art of pharmacy. Such methods include the step of bringing into association the active ingredient with the carrier which constitutes one or more accessory ingredients or excipients. In general, the formulations are prepared by uniformly and intimately bringing into association the active ingredient with liquid carriers or finely divided solid carriers or both, and then, if necessary, shaping the product. For example, a tablet is made by compression or molding, optionally with one or more accessory ingredients. Compressed tablets may be prepared by compressing in a suitable machine TP or its salt in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Smoking and hormones are two important etiological factors involved in breast cancer formation. The invention demonstrates that EGCG prevents the binding of nicotine (Nic) (especially high concentrations of nicotine (10 μM)) to its receptor. The results are of clinical importance because a previous report demonstrated that the steady-state serum concentration of nicotine in smokers was 200 nM and dramatically increased to 10-100 μM in serum or even to 1 mM in the saliva immediately after smoking (West, K. A., Brognard, J., Clark, A. S., Linnoila, I. R. et al., J. Clin. Invest. 2003, 111, 81-90). Although many in vitro and animal studies have suggested a protective effect of green tea against breast cancer, findings from epidemiological studies have been inconsistent (Iwasaki, M., Inoue, M., Sasazuki, S., Miura, T. et al., Breast Cancer Res. Treat. 2010; Nakachi, K., Suemasu, K., Suga, K., Takeo, T. et al., Jpn. J. Cancer Res. 1998, 89, 254-261). For example, the possible protective role of EGCG in human breast cancer formation was investigated recently (Iwasaki, M., Inoue, M., Sasazuki, S., Miura, T. et al., Breast Cancer Res. Treat. 2010). A long-term study (10.6 years) with a large cohort (24226 women aged 40-69 years) was performed to explore the relationship between plasma levels of tea polyphenols, including (−)-epigallocatechin (EGC), (−)-epicatechin (EC), EGCG, and (−)-epicatechin-3-gallate (ECG), and breast cancer risk. The results did not support the hypothesis that tea polyphenols have protective effects. The invention indicates that pretreatment with EGCG profoundly inhibited [³H]-Nic binding activity in wild-type MCF-7 cells and further demonstrates that siRNA knockdown of α9-nAChR significantly (>40%) inhibits [³H]-Nic binding activity. Pretreatment with EGCG profoundly (>90%) inhibited [³H]-Nic binding activity in (Si) cells when compared with Sc or wild-type cells. In addition, E2- or Nic-induced overexpression of the a9-nAChR protein was almost completely inhibited in EGCG-pretreated cells. By the same principle, TP or a pharmaceutically acceptable salt thereof provides effects in aiding smoking cessation and/or treating and/or preventing smoking addition.

The invention suggests that diet-based protection against breast cancer may partly derive from synergy amongst dietary phytochemicals directed against specific molecular targets in responsive breast cancer cells. The invention also provides support for the feasibility of the development of a diet-based combinatorial approach in the prevention and treatment of breast cancer. Importantly, the invention provides evidence that E2 significantly increases the a9-nAchR protein expression levels in breast cells.

### EXAMPLE

### Pharmacological Assay

### Chemicals

Curcumin was purchased from E. Merck (Darmstadt, Germany). Magnolol, dibenzoylmethane, EGCG, 1-(2-hydroxyphenyl)-3-phenyl-1,3-propanedione and 1-(2-hydroxy-5-methylphenyl)-3-phenyl-1,3-propanedione were purchased from Sigma-Aldrich Chemical (Milwaukee, Wis., USA). Aqueous stock solutions of 10 mM Nic and NNK (Chemsyn, Lenexa, Kans., USA) were prepared in double-distilled H2O. The other chemicals used in this study were dissolved in DMSO as described in Pan, M. H., Huang, M. C., Wang, Y. J., Lin, J. K., Lin, C. H., J. Agric. Food Chem. 2003, 51, 3977-3984 and Chen, L. C., Liu, Y. C., Liang, Y. C., Ho, Y. S., Lee, W. S., J. Agric. Food Chem. 2009.

### Cell Culture

Human mammary gland epithelial adenocarcinomas (MCF7, MDA-MB-231, AU-565, MDA-MB-453, and BT-483) and normal human mammary gland epithelial fibrocystic cell lines (MCF-10A and HBL-100) were purchased from ATCC (American Type Culture Collection, Manassas, Va., USA). MCF-7, MDA-MB-231, and MDA-MB-453 cells were maintained in DMEM, whereas BT-483 cells were maintained in RPMI-1640. Cell growth, proliferation, and viability were determined by the 3-(4,5-dimethylthiazol-2-yl)-2,5-diphenyltetrazolium (MTT) assay. The formazan product was measured by absorbance at 540 nm.

### RNA Isolation and RT-PCR Analysis

Total RNA was isolated from human cell lines using TRIzol (Invitrogen, Carlsbad, Calif., USA) according to the manufacturer's protocol. The α9-nAChR subunit-specific primers were synthesized by MB Mission BioTech (Taipei, Taiwan) as described in the previous reports (Chen, C. S., Lee, C. H., Hsieh, C. D., Ho, C. T. et al., Breast Cancer Res. Treat. 2010; Shih, Y. L., Liu, H. C., Chen, C. S., Hsu, C. H. et al., J. Agric. Food Chem. 2010, 58, 235-241). The PCR amplicons were analyzed on 1.2% agarose gels (Amresco, Solon, Ohio, USA) stained with ethidium bromide. The α9-nAChR mRNA intensity was measured and normalized to β-glucuronidase expression. Pictures of the bands were taken using an INFINITY-α digital imaging system (Vilber Lourmat, France), and band intensities were determined using PhotoCapt Version 11.01 software.

### Protein Extraction, Western Blotting, and Antibodies

To prepare protein samples, cells were washed once with ice-cold PBS and lysed on ice in cell lysis buffer (50 mM Tris-HCl, pH 8.0, 120 mM NaCl2, 0.5% Nonidet P-40, 100 mM sodium fluoride, and 200 μM sodium orthovanadate) containing protease inhibitors as described previously (Shih, Y. L., Liu, H. C., Chen, C. S., Hsu, C. H. et al., J. Agric. Food Chem. 2010, 58, 235-241). Protein (50 μg) from each sample was resolved by 12% SDS-polyacrylamide gel electrophoresis, transferred, and analyzed by Western blotting. Antibodies were purchased from the following vendors: anti-total AKT and protein A/G agarose beads were obtained from Santa Cruz Biotechnology (Santa Cruz, Calif., USA); anti-GAPDH and anti-α9-nAChR antibodies were from Abcam (Cambridge, UK); antiphospho AKT (Ser⁴⁷³), anti-c-Jun, and anti-c-Fos were from Cell Signaling Technology (Beverly, Mass., USA). The secondary antibodies, alkaline phosphatase-coupled anti-mouse, and anti-rabbit IgG were purchased from Santa Cruz Biotechnology. The specific protein complexes were identified with the chromogenic substrates nitro blue tetrazolium, and 5-bromo-4-chloro-3-indolyl-phosphate from KPL (Gaithersburg, Md., USA). In each experiment, proteins were also probed with anti-GAPDH antibodies as a protein-loading control.

### Soft Agar Cloning Assay

The base layer consisted of 0.9% low gelling point SeaPlaque agarose (Sigma, St. Louis, Mo., USA) in culture medium (Shih, Y. L., Liu, H. C., Chen, C. S., Hsu, C. H. et al., J. Agric. Food Chem. 2010, 58, 235-241). Soft agar, consisting of 0.4% SeaPlaque agarose in culture medium, was mixed with 1×10⁴ MCF-7 cells and plated on top of the base layer in 60-mm diameter culture dishes. Soft agar cultures were maintained at 37° C. and counted for the appearance of colonies with a Leica DMI 4000B Microscope Imaging System (Leica Microsystems, Wetzlar, Germany).

### RNA Interference

Expression of α9-nAChR was ablated in breast cancer cells with at least two independent small interfering RNAs (siRNAs) (Chen, C. S., Lee, C. H., Hsieh, C. D., Ho, C. T. et al., Breast Cancer Res. Treat. 2010). The target sequences of α9-nAChR mRNA were selected to suppress α9-nAChR gene expression. Scrambled sequences of each siRNA were used as controls (Chen, C. S., Lee, C. H., Hsieh, C. D., Ho, C. T. et al., Breast Cancer Res. Treat. 2010). After BLAST analysis to verify that there were no significant sequence homologies with other human genes, the selected sequences were inserted into BglII/HindIII digested pSUPER vectors to generate the pSUPER-Si α9-nAChR and pSUPER-scramble vectors. All constructs were confirmed by DNA sequence analysis. The transfection protocol utilized has been described previously (Chen, C. S., Lee, C. H., Hsieh, C. D., Ho, C. T. et al., Breast Cancer Res. Treat. 2010; Shih, Y. L., Liu, H. C., Chen, C. S., Hsu, C. H. et al., J. Agric. Food Chem. 2010, 58, 235-241). Briefly, 1.5×10⁵ cells were washed twice with PBS and mixed with 0.5 μg of plasmid. Then, one pulse was applied for 20 ms under a fixed voltage of 1.6 kV on a pipette-type MP-100 microporator (Digital Bio, Seoul, Korea).

Generation of Stable nAChR-Knockdown Cell Lines

At least three clones of the MCF-7 cell lines stably expressing the siRNA were generated. All experiments were performed using multiple subclones of each cell line with consistent results. The pSUPER-Si α9-nAChR and pSUPER-scramble vectors were transfected and stable integrants were selected 72 h later with Geneticin (4 mg/mL). After 30 days in selective medium, two geneticin (G418)-resistant clones, referred to as pSUPER-Si α9-nAChR, were isolated. These clones demonstrated 480% reduction in mRNA and protein levels when compared with the control clones (pSUPER-scramble).

### Plasmid Constructions

All α9-nAChR promoter—luciferase gene fusions were made in the pGL3-Basic vector (Promega, Madison, Wis., USA) by introducing the appropriate α9-nAChR promoter fragments into the polylinker upstream of the luciferase gene. These fragments were generated with restriction enzymes and cloned directly into pGL3-Basic or subcloned first in pBluescript and then transferred to pGL3-Basic. Deletion analysis of the most promoter-proximal region was performed by generating either appropriate restriction enzyme fragments or PCR fragments with suitable sense oligonucleotides and an antisense primer (5′-TATAGAGGCTCAGGAAAAAG-3′ (SEQ ID NO:1)) that anneals to the pGL3-Basic vector downstream of the site of transcription initiation.

### Luciferase Activity Assay

MCF-7 cells were plated in 6-well plates. Cells were transiently cotransfected the next day with 1.5 μg of PGL3-Basic plasmid containing the full-length α9-nAChR promoter and 0.1 μg of the RLTK plasmid (Promega) using an MP-100 microporator (Digital Bio), according to the manufacturer's instructions. After incubation for 24 h, the medium was changed to culture medium containing 10% FBS or 0.1% FB S with or without Nic. Cells were lysed 24 h later with 1× Reporter Lysis Buffer (Promega) and stored frozen at −20° C. overnight. Luciferase activity was determined by testing 50 μL of the cell lysate and 50 μL of the Luciferase Assay Reagent (Promega) using a HIDEX Chameleon Microplate Reader. Relative luciferase units were normalized to renilla luciferase from the same cell lysates. Each luciferase assay experiment was performed three times.

### Chromatin Immunoprecipitation Assay

Chromatin immunoprecipitation (ChIP) assays of cultured cells were performed as described previously (Chen, C. S., Lee, C. H., Hsieh, C. D., Ho, C. T. et al., Breast Cancer Res. Treat. 2010; Shih, Y. L., Liu, H. C., Chen, C. S., Hsu, C. H. et al., J. Agric. Food Chem. 2010, 58, 235-241). Briefly, after various regimens of Nic treatment, the cells were fixed with a final concentration of 1% formaldehyde directly added to the cell culture medium at 25° C. for 15 min. The crosslinking reaction was terminated with 0.125 M glycine for 5 min, and the cells were collected into a new Eppendorf tube. The cell lysate was sonicated three times with 10 s bursts to yield input DNA enriched for fragments around 1000 bp in size. A c-Jun (AP-1) antibody (Santa Cruz Biotechnology) was used for the immunoprecipitation reactions. Primers, which detect the a9-nAChR promoter regions, amplified a region from −960 to −1 for 32 cycles (forward: 5′-ATGCAATG CAAGCCTGAGCT-3′ (SEQ ID NO:2) and reverse: 5′-TATA-GAGGCTCAGGAAAAAG-3′ (SEQ ID NO:3)). The PCR products were then separated and analyzed by agarose gel electrophoresis.

### [³H]-Nic Equilibrium Binding

L-(−)-[N-methyl-³H]-Nic ([³[H]-Nic) (71-75 Ci/mmol) was purchased from Dupont/NEN Research Products (Boston, Mass., USA), and free-base Nic (99% pure) was purchased from The Eastman Kodak (Rochester, N.Y., USA). To study the uptake of [³H]-Nic in MCF-7 cell monolayers (2×10⁶ cells/well), cells were rinsed three times with a buffer containing 140 mM NaCl, 5.4 mM KCl, 1.8 mM CaCl2, 0.8 mM Mg504, 5 mM glucose, and 25 mM Hepes/Tris (pH 7.4). Saturation binding studies were conducted in 6-well plates using at least eight different concentrations of [³H]-Nic ranging from 1 to 15 nM for 2 h at 37° C. Nonspecific binding was determined by the addition of 10 nM Nic. Total binding was evaluated for each concentration in quintuplicate, and blanks were determined in triplicate. The uptake of [³H]-Nic was halted by aspiration of the uptake medium and washing of the wells three times with ice-cold buffer. The cells were lysed in 1 mL of 0.5% Triton X-100, and aliquots of the cell lysates were transferred to scintillation vials to determine the incorporated radioactivity by scintillation counting. Inhibition studies were carried out by adding various concentrations of unlabeled compounds to the uptake medium.

### Statistics

All data are expressed as the mean value±SE. Comparisons were subjected to a one-way analysis of variance (ANOVA) followed by Fisher's least significant difference test. Significance was set at p<0.05.

### EXAMPLE 1

### Nic- and E2-Induced nAChR Expression in a Human Breast Cancer Cell Line

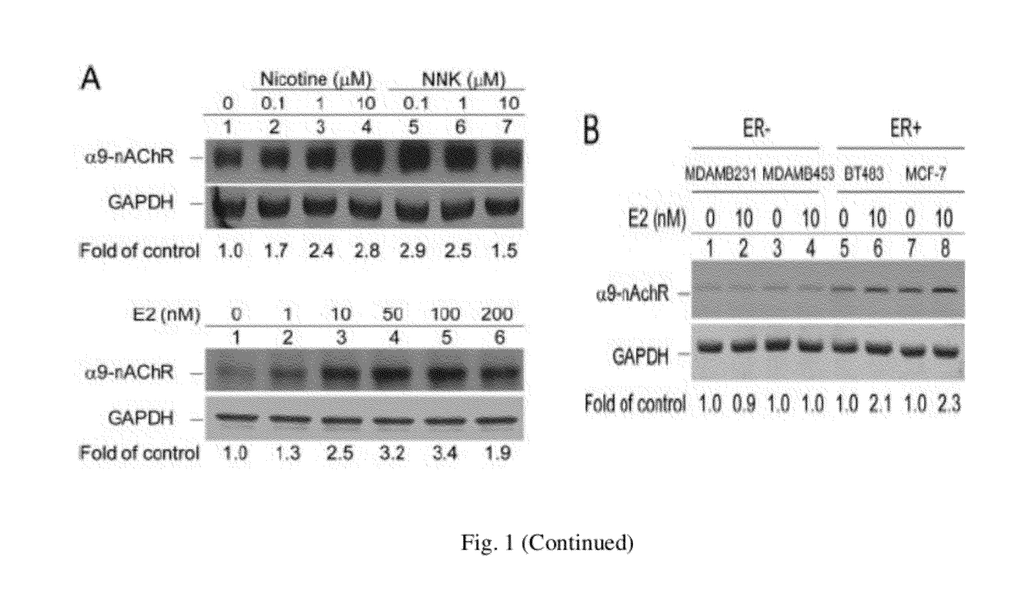

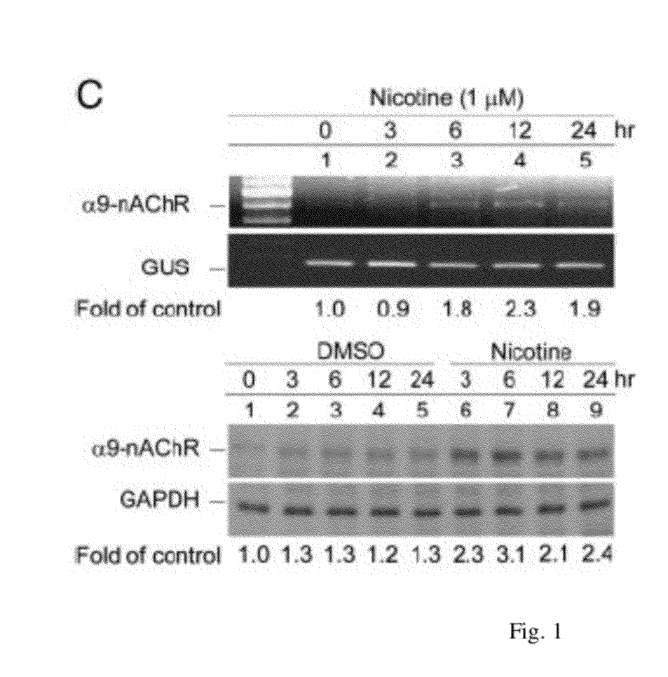

Smoking and hormones are two important etiological factors involved in breast cancer formation (Daniell, H. W., breast cancer, and smoking. N. Engl. J. Med. 1980, 302, 1478). A recent study revealed that the expression of the α9-nAChR subunit in MCF7 cells is largely responsible for the observed Nic-induced proliferation (Chen, C. S., Lee, C. H., Hsieh, C. D., Ho, C. T. et al., Breast Cancer Res. Treat. 2010). To test whether nicotinic-induced cell proliferation signaling was also influenced by crosstalk from hormones (such as E2) through the induction of α9-nAChR, MCF-7 cells were treated with different doses of Nic, its metabolically active carcinogenic compound NNK, and E2 (FIG. 1A). The results demonstrate that the α9-nAChR protein level was significantly induced in MCF-7 cells by either smoking components (NNK, Nic) or the hormone (E2) in a dose-dependent manner (FIG. 1A). To test whether the α9-nAChR protein induction in E2-induced MCF-7 cells was ER status specific, human breast cancer cells with (MCF-7 and BT483) or without (MDA-MB-231 and MDA-MB-453) ER expression were treated with E2 (10 nM) for 24 h (FIG. 1B). The E2-induced α9-nAChR protein induction was clearly detected in the ER+(MCF-7 and BT483) cells compared with the ER (MDA-MB-231 and MDA-MB-453) cells (FIG. 1B). Time course experiments demonstrated that significant increases in the α9-nAChR mRNA and protein levels in MCF-7 cells were detected 6 h after Nic treatment (FIG. 1C).

### EXAMPLE 2

### Nic- and E2-Induced a9-nAChR Expression was Blocked by the Tea Polyphenol (EGCG) in MCF-7 Cells

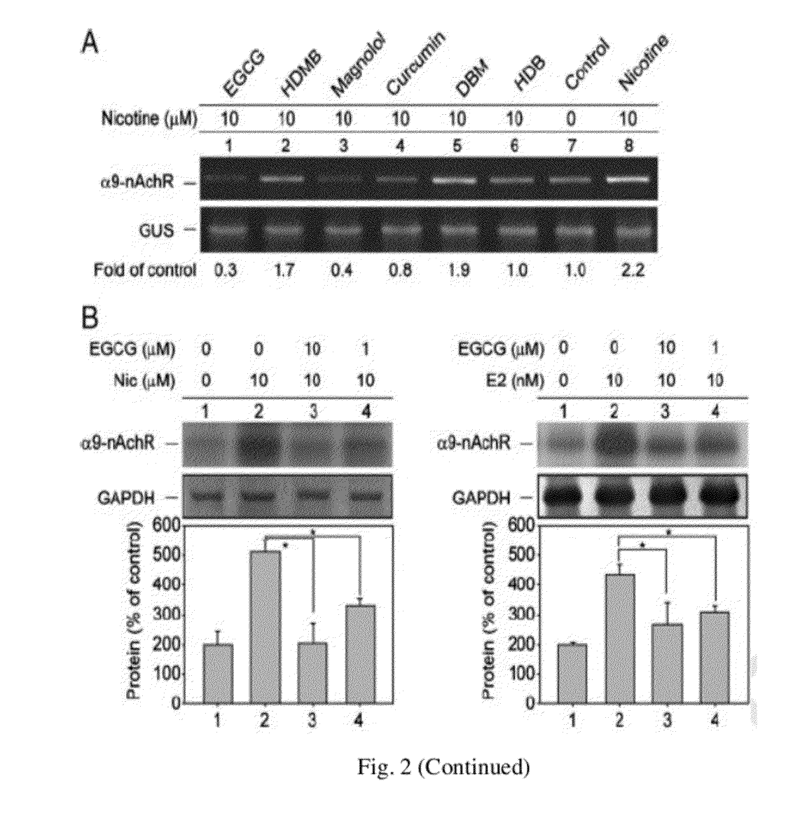

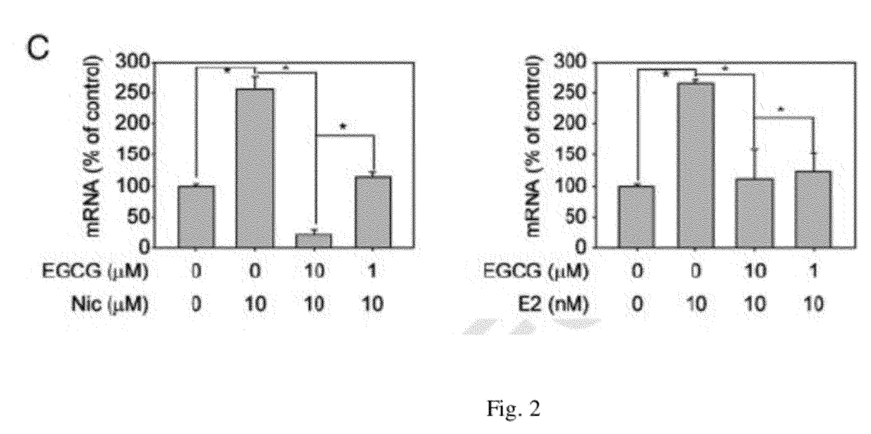

Several natural polyphenolic compounds derived from edible plants were evaluated. All these compounds have been reported in previous articles to have antiproliferative or apoptosis-inducing activities in human cancer cells (Pan, M. H., Huang, M. C., Wang, Y. J., Lin, J. K, Lin, C. H., J. Agric. Food Chem. 2003, 51, 3977-3984; Aggarwal, B. B., Sethi, G., Ahn, K. S., Sandur, S. K et al., Ann. NY Acad. Sci. 2006, 1091, 151-169). It was found that α9-nAChR mRNA and protein were downregulated by EGCG (1 μM) in Nic-treated cells (FIGS. 2A and B). It was then tested whether E2-induced overexpression of α9-nAChR could also be blocked by EGCG. MCF-7 cells were treated with E2 in the presence or absence of EGCG (FIG. 2B). The results demonstrate that αa9-nAChR protein expression, which was induced by either Nic or E2, was significantly inhibited by EGCG in MCF-7 cells (FIG. 2B). In order to evaluate the quantitative effects of EGCG-induced α9-nAChR gene downregulation, we performed additional real-time PCR experiments to detect the α9-nAChR mRNA expression level in the EGCG- and Nic-treated human breast cancer cells. The results demonstrated that the α9-nAChR mRNA expression was significantly upregulated in response to Nic (10 μM) or E2 (10 nM) for 24-h treatment (FIG. 2C, left and right figures, bars 1 versus 2; *p<0.05). However, such effects were completely attenuated in the EGCG (10 μM)-treated cells (FIG. 2C, left and right figures, bars 2 versus 3; *p<0.05). Such results demonstrated that the Nic-induced α9-nAChR cell growth proliferation signal was attenuated by EGCG through downregulation of α9-nAChR mRNA expression.

### EXAMPLE 3

### EGCG Pretreatment Attenuated Nic-and E2-Induced a9-nAChR Protein Expression through Inhibition of AKT Signals in MCF-7 Cells

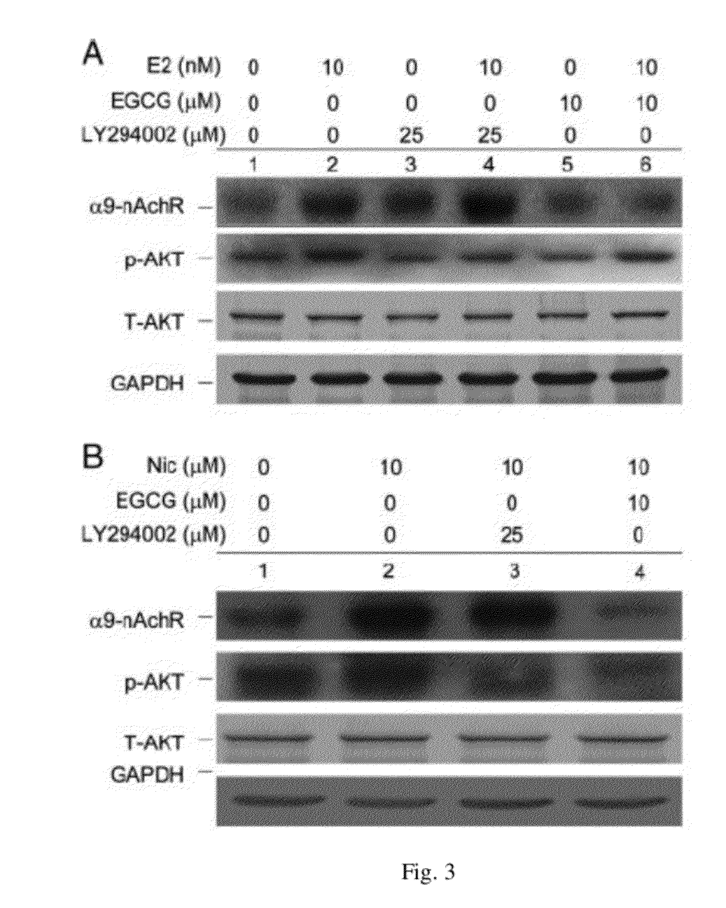

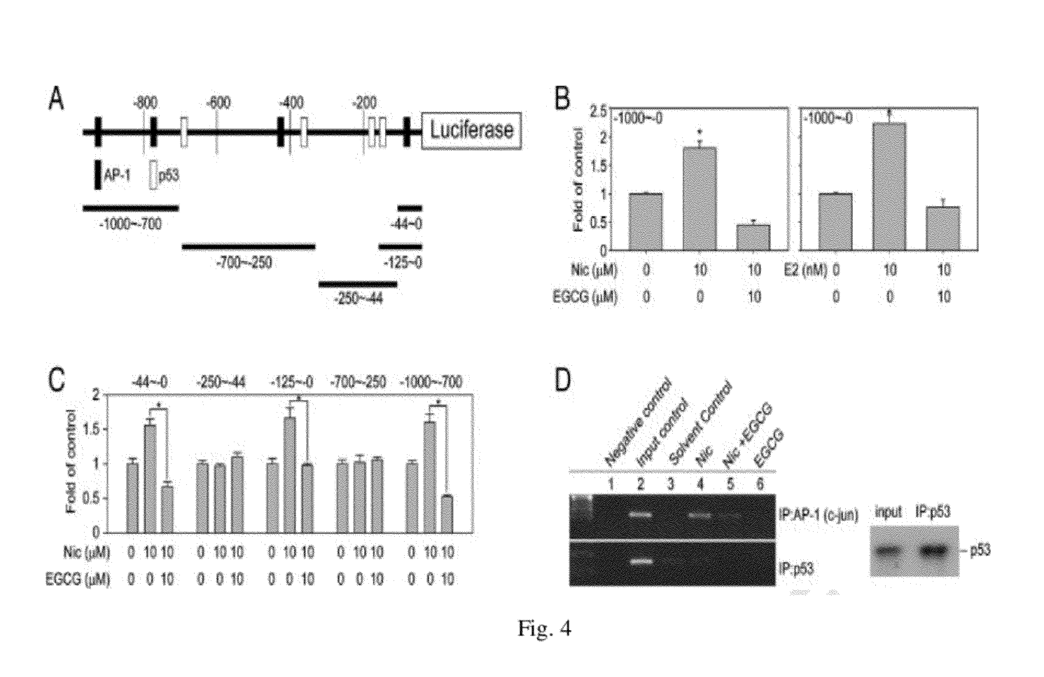

A previous study demonstrated that AKT is activated through phosphorylation of serine (Ser⁴⁷³) or threonine (Thr³⁰⁸) by different upstream stimulatory factors involved in breast cancer cell proliferation (Liu, Z., Yu, X., Shaikh, Z. A., Toxicol. Appl. Pharmacol. 2008, 228, 286-294). To test whether PI3 kinase (PI3K)-mediated signals are involved in Nic- and E2-induced α9-nAChR activation, MCF-7 cells were pretreated with a PI3K/AKT inhibitor (LY294002, 25 μM) or EGCG (10 μM) for 24 h and were subsequently treated with Nic (10 μM) or E2 (10 nM) for an additional 24 h. Immunoblotting analysis was performed, and the results show that Nic and E2 treatment significantly increased the levels of p-AKT (Ser⁴⁷³) (FIGS. 4A and B, lane 2). However, our study shows that Nic- and E2-induced upregulation of α9-nAChR is not significantly inhibited by LY294002 through inactivation of pAKT (FIG. 3A, lane 4 and FIG. 3B, lane 3). On the contrary, both α9-nAChR and pAKT protein expressions, which were induced by either E2 or Nic, were significantly inhibited by EGCG (FIG. 3A, lane 6 and FIG. 3B, lane 4). This result indicates that other signaling proteins are involved in the transcriptional activation of α9-nAChR protein expression.

### EXAMPLE 4

### Identification of Transcription Factor Responses to EGCG-Attenuated Nic-and E2-Induced α9-nAChR Protein Expression in MCF-7 Cells

To explore this hypothesis, a 1-kb genomic fragment encompassing the full-length human α9-nAChR promoter was inserted upstream of a luciferase reporter gene (FIG. 4A). Luciferase activity obtained with the full-length α9-nAChR promoter was compared with that obtained with the vector alone and defined as 100% (baseline level). Experiments utilizing the full-length construct demonstrated that a9-nAChR promoter activity in MCF-7 cells was significantly induced by 24-h treatment with Nic (10 μM) or E2 (10 nM) (41.8 and 2.3-fold, respectively) (FIG. 4B, *p<0.05). MCF-7 cells were also pretreated with EGCG (10 μM) for 24 h and then treated with Nic (or E2) for an additional24 h. The results reveal that pretreatment with EGCG significantly reduces the Nic- and E2-induced α9-nAChR promoter luciferase activity (FIG. 4B, *p<0.05).

To define more precisely the regulatory elements that the α9-nAChR promoter required for Nic-induced transcriptional activation, transient transfection experiments were conducted with a series of 5′ promoter deletion constructs subcloned into the pGL3 vector. The resulting plasmids were transfected into MCF-7 cells. Specific response elements in the α9-nAChR promoter were eliminated in the shorter constructs. The Nic-induced responsiveness was preserved when the promoter fragments were constructed as pGL3(−44-0), pGL3(−125-0), and pGL3(−1000-700) (FIG. 4C). These results suggest that the Nic-induced transcription factor responsive elements were located at the AP-1 and p53 sites (FIG. 4A).

To determine whether the AP-1 or p53 transcription factors bind directly to the relevant α9-nAChR promoter-binding sites in response to Nic treatment, ChIP assays were performed in MCF-7 cells. The results demonstrate that increased binding of AP-1 to the α9-nAChR promoter was detected 24 h after Nic (10 μM) treatment in MCF-7 cells (FIG. 4D, lane 4). Pretreatment with EGCG (10 μM, 24 h) attenuated the Nic-induced AP-1 binding to the α9-nAChR promoter (FIG. 4D, lanes 5 versus 4). Interestingly, we found that p53 is a transcription factor that binds to the a9-nAChR promoter (−125 to 0) and is not affected by EGCG (FIG. 4D, lanes 5 versus 4).

### EXAMPLE 5

### EGCG Affects Nic-Induced Breast Cancer cell Proliferation through the Inhibition of α9-nAChR-Mediated Signals

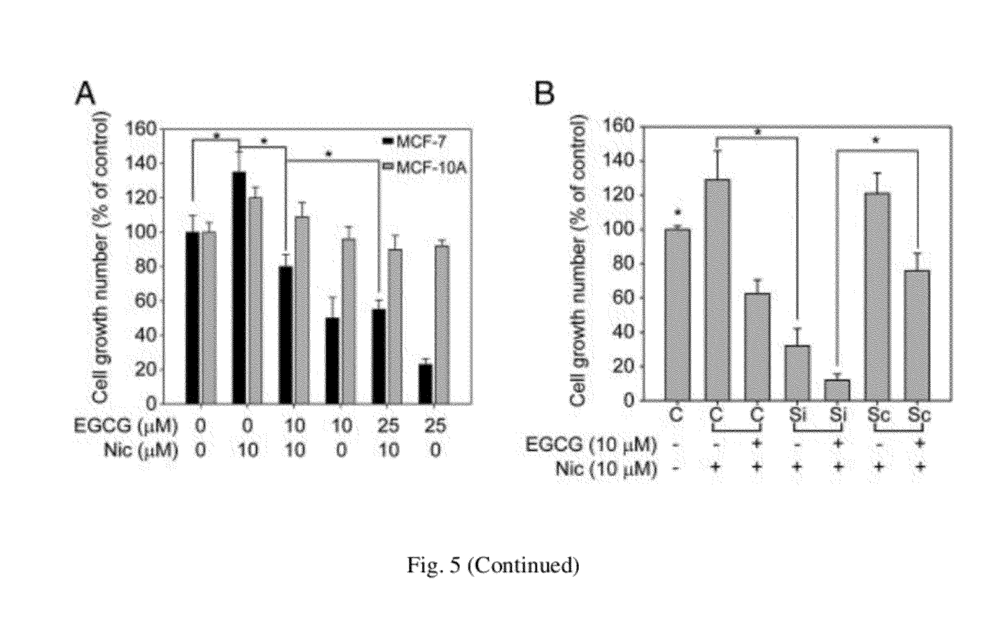

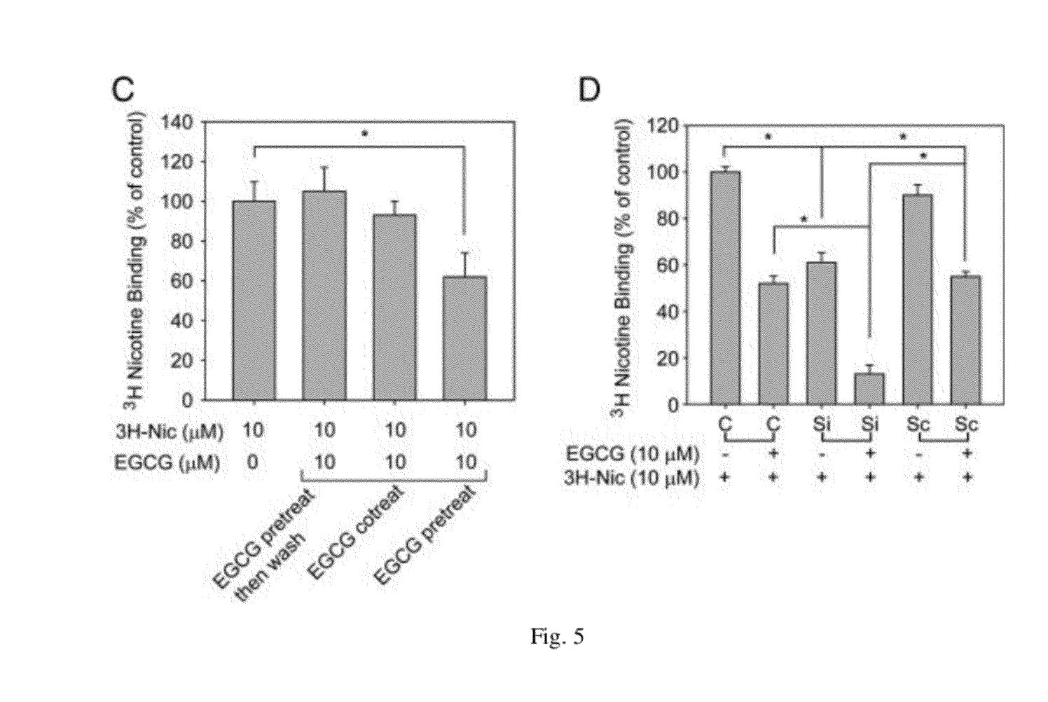

To test whether α9-nAChR in human breast cancer cells could be used as a therapeutic molecular target, human breast cancer (MCF-7) and normal (MCF-10A) cells were treated with Nic (10 μM) in the presence or absence of EGCG (10-25 μM, 24 h) pretreatment, and the cell number was counted. It was found that Nic significantly stimulated breast cancer cell growth, and this proliferation was blocked by EGCG in a dose-dependent manner (FIG. 5A, bars 3 versus 5 and bars 5 versus 9; *p<0.05). In contrast, no changes in cell number by either Nic or EGCG treatment in normal breast epithelial (MCF-10A) cells were observed (FIG. 5A). To explore the possible role of Nic-induced expression of the α9-nAChR subunit in human breast cell proliferation, a stable MCF-7 cell line was established (Lee, C. H., Huang, C. S., Chen, C. S., Tu, S. H. et al., J. Natl. Cancer Inst. 2010, in press) in which the expression of a9-nAChR was knocked down by RNA interference (FIG. 5B, bars 4 and 5). The cellular proliferation rates in wild-type MCF-7 cells (C) and cells containing the scrambled vector control (Sc) were significantly increased by treatment with Nic (10 μM) (FIG. 5B, bars 1 versus 2 and bars 1 versus 6; *p<0.05). The EGCG (10 μM, 24 h) pretreatment reversed the Nic-induced cellular proliferation effects (FIG. 5B, bars 3 versus 2 and bars 7 versus 6; *p<0.05). However, the growth effects observed in Nic-induced cells were almost completely attenuated in a9-nAChR siRNA knockdown (Si) cells (FIG. 5B, bars 4 versus 2). Interestingly, combined treatment with EGCG profoundly inhibited cell proliferation in the Nic-treated a9-nAChR (Si) cells when compared with the Sc cells (FIG. 5B, bars 5 versus 7; *p<0.05).

These results suggest that breast cancer cell proliferation induced by Nic could be mediated through endogenous α9-nAChR. Therefore, agents that block the binding of Nic to α9-nAChR should be able to block the Nic-mediated cell proliferation effects. To test this hypothesis, MCF-7 cells were treated with [³H]-Nic to determine its ligand-receptor binding activity. Previous results demonstrated that the dissociation constant (Kd) of [³H]-Nic binding is 3 nM, and that its maximum binding activity is attained at 60 min in MCF-7 cells (Lee, C. H., Huang, C. S., Chen, C. S., Tu, S. H. et al., J. Natl. Cancer Inst. 2010, in press). The relative [³H]-Nic binding activities were detected in cells with different treatment regimens: 24 h [³H]-Nic treatment (control group); [³H]-Nic+EGCG cotreatment for 24 h (cotreat group); EGCG pretreatment for 24 h followed by [³H]-Nic cotreatment for an additional 24 h (pretreat group); or EGCG pretreatment for 24 h, followed by washing and then [³H]-Nic treatment for an additional 24 h (pretreat, then wash group). The results demonstrate that the relative [³H]-Nic binding activity is significantly blocked in the EGCG pretreated group (FIG. 5C, bars 1 versus 4; *p<0.05). In contrast, the [³H]-Nic binding activity was not significantly inhibited in the EGCG cotreated or the pretreated, then washed groups (FIG. 5C, bars 1 versus 2 and bars 1 versus 3). In addition, the mean [³H]-Nic binding activity was significantly inhibited in MCF-7 (Si) cells compared with wild-type (C) or to (Sc) cells (FIG. 5D, bars 3 versus 1 or 3 versus 5; “p<0.05). Interestingly, pretreatment with EGCG profoundly inhibited the [³H]-Nic binding activity in the Nic-treated α9-nAChR (Si) cells when compared with the Sc or wild-type cells (FIG. 5D, bars 4 versus 2 or 4 versus 6; “p<0.05).

### EXAMPLE 7

### Effects of EGCG on the Inhibition of Nic-Induced Breast Cancer Cell Colony Formation in Soft Agar

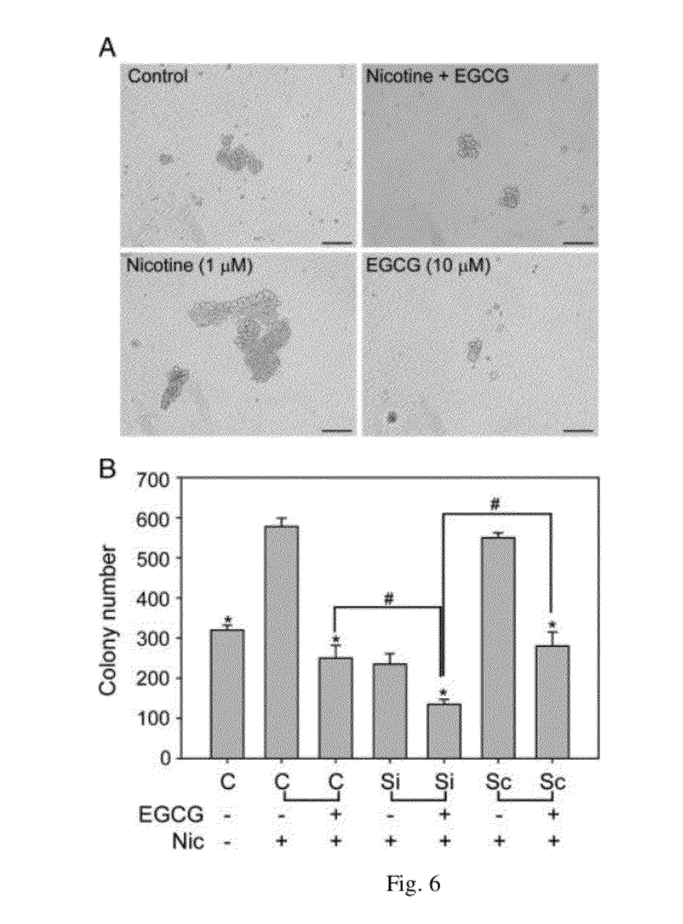

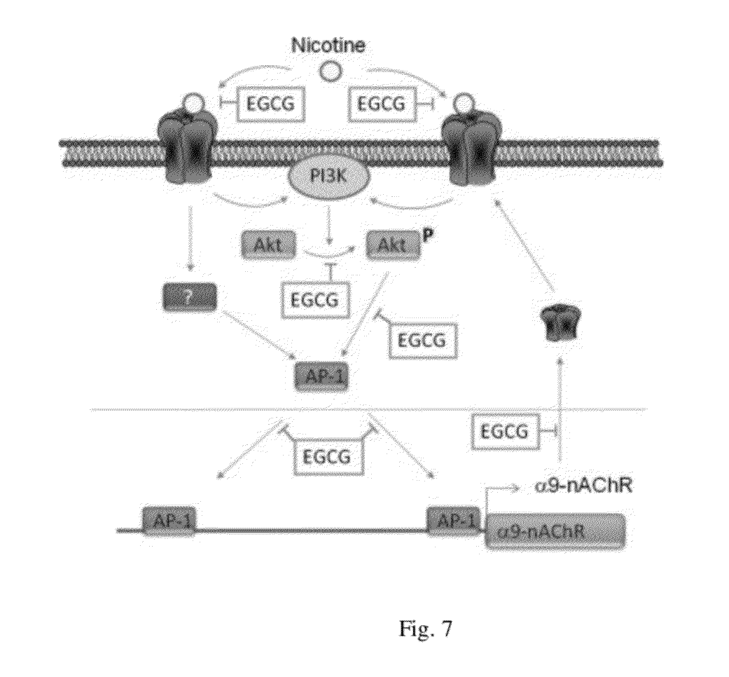

Previous studies using a soft agar transformation assay and a xenografted nude mouse animal model demonstrated the transformation of noncancerous human breast epithelial (MCF-10A) cells by either a cigarette smoke condensate or the tobacco-specific carcinogen, NNK (Mei, J., Hu, H., McEntee, M., Plummer, H., 3rd et al., Breast Cancer Res. Treat. 2003, 79, 95-105; Siriwardhana, N., Choudhary, S., Wang, H. C., Breast Cancer Res. Treat. 2008, 109, 427-441). To test whether Nic-induced formation of colonies are through the activity of its cognate receptor (α9-nAChR), soft agar assays were performed with human breast cancer cells (MCF-7). Cells, α9-nAChR (Si) and (Sc), were treated with Nic (10 μM) in the presence or absence of EGCG (10 μM) (FIGS. 6A and B). The results show that EGCG (10 μM) significantly inhibits the formation of transformed colonies when compared with Nic-treated cells (FIG. 6B, bars 2 versus 3, “p<0.05). Interestingly, treatment with EGCG profoundly inhibited colony formation in a9-nAChR (Si) cells when compared with (Sc) cells (FIG. 6B, bars 5 versus 3 and 5 versus 7, #p<0.05).

What is claimed is:

1. A method of treating and/or preventing nicotine or nicotine-derived compounds or estrogen induced breast cancer, which comprises administering to a subject an effective amount of a tea polyphenol or a pharmaceutically acceptable salt thereof, thereby treating and/or preventing nicotine or estrogen induced breast cancer.

2. The method of claim 1, wherein the nicotine or estrogen induced breast cancer is caused by E2- or Nic-induced expression of the α9-nAChR protein.

3. The method of claim 2, wherein the expression α9-nAChR is overexpressed.

4. The method of claim 1, which is for treating and/or preventing smoking-mediated breast tumorigenesis.

5. The method of claim 1, wherein the method is for preventing nicotine or estrogen induced breast cancer.

6. The method of claim 1, wherein the method is for providing chemopreventive ability.

7. The method of claim 1, wherein the TPs are catechins.

8. The method of claim 6, wherein the catechins are selected from the group consisting of epigallocatechin gallate (EGCG), epigallocatechin (EGC), epicatechin gallate (ECG), epicatechin (EC), gallocatechin gallate (GCG), gallocatechin (GC), catechin gallate (CG) and catechin (C).

9. The method of claim 8, wherein the catechin is EGCG.

10. The method of claim 9, wherein EGCG is (−)-EGCG.

11. The method of claim 1, wherein TP or salts thereof may be used in combination therapies selected from the group consisting of surgery, radiation therapy, chemotherapy, cytotoxic agents, cytostatic agents and hormone therapies.

12. The method of claim 1, wherein the administration is oral, rectal, parenteral, transdermal, transmucosal, subcutaneous, intramuscular, intrathecal, intrapulmonary, nasal or vaginal.

13. The method of claim 1, wherein the amount administered to the subject is from about 500 to about 2500 mg/kg body weight.

14. The method of claim 1, wherein the treatment and/or prevention is for nicotine induced breast cancer.

15. The method of claim 1, wherein the treatment and/or prevention is for estrogen induced breast cancer.

16. The method of claim 1, wherein TP or salts thereof may be used in combination with chemotherapy, surgery, radiation, and hormone therapy.

17. The method of claim 1, wherein the nicotine-derived compound is NNN (“N′-nitrosonomicotine”), NNK (“4-(methylnitros-amino}-t-(3-pyridyl)-1-butanone” or NNA (“4-fmethylnitrosamino)-4-{3-pyridyl)-butanal “).

18. A method of aiding smoking cessation and/or treating and/or preventing smoking addition, which comprises administering to a subject an effective amount of a tea polyphenol or a pharmaceutically acceptable salt thereof, thereby aiding smoking cessation and/or treating and/or preventing smoking addition.

19. The method of claim 18, wherein the TPs are catechins.

20. The method of claim 19, wherein the catechins are selected from the group consisting of epigallocatechin gallate (EGCG), epigallocatechin (EGC), epicatechin gallate (ECG), epicatechin (EC), gallocatechin gallate (GCG), gallocatechin (GC), catechin gallate (CG) and catechin (C).

21. The method of claim 19, wherein the catechin is EGCG.

22. The method of claim 21, wherein EGCG is (−)-EGCG.

23. The method of claim 18, wherein the administration is oral, rectal, parenteral, transdermal, transmucosal, subcutaneous, intramuscular, intrathecal, intrapulmonary, nasal or vaginal.

24. The method of claim 18, wherein the administration is through a transdermal patch or inhaler.
